(19) United States
(12) Patent Application Publication
(10) Pub. No.: US 2010/0015628 A1
(43) Pub. Date: Jan. 21, 2010

(54) AMBIENT TEMPERATURE STABLE CHEMICAL/BIOLOGICAL REAGENTS ON MEMBRANES OR FILTERS

(75) Inventors: Joseph W. Farchaus, Bloomsbury, NJ (US); Reddy Ponaka, Dayton, NJ (US)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES CORP., Piscataway, NJ (US)

(21) Appl. No.: 12/523,779

(22) PCT Filed: Feb. 20, 2008
(86) PCT No.: PCT/US2008/054343

§ 371 Date: Jul. 20, 2009

Related U.S. Application Data
(60) Provisional application No. 60/891,946, filed on Feb. 28, 2007.

(51) Int. Cl. C12Q 1/68

(52) U.S. Cl. 435/6

(57) ABSTRACT

The present invention provides a biological sample preparation system including ambient temperature stable reagent mixture and a separation filter or membrane. In particular, the system includes a dried reagent in a glassy, porous state, on top of a separation column, whereby sample preparation is streamlined and simplified. Also provided are methods of making and using the system. A kit for preparing a biological sample is also provided.

### CROSS-REFERENCE TO RELATED APPLICATIONS

This application is a filing under 35 U.S.C. § 371 and claims priority to international patent application number PCT/US2008/054343 filed Feb. 20, 2008, published on Oct. 2, 2008, as WO 2008/118566, which claims priority to U.S. provisional patent application No. 60/891,946 filed Feb. 28, 2007; the disclosure of which is incorporated herein by reference in its entirety.

### FIELD OF THE INVENTION

This invention relates to a biological sample preparation system including ambient temperature stable reagent mixture and a separation filter or membrane. In particular, it relates to dried reagent in a glassy, porous state, on top of a separation column, whereby sample preparation is streamlined and simplified.

### BACKGROUND OF THE INVENTION

Modern molecular biology routinely requires the separation of biological macromolecules from the source material such as bacterium, plant or animal tissues or cells. Many of the separation processes include lysis of the cells followed by affinity separation through a membrane or column device. These processes usually involve multiple reagents and a number of steps, thus can be tedious, time consuming, and error prone. In addition, some of the source material (body fluid or environmental hazards/pathogen) pose a threat to the researcher, and minimum handling is desirable.

For example, many applications of modem molecular biology require the isolation of DNA from a source comprising mixtures of the DNA with heterologous material such as proteins, lipids and other cellular constituents. Particularly important examples of such heterogeneous mixtures include plant and animal tissues and cells, cleared bacterial or yeast lysates containing plasmid or cosmid DNA, recombinant phage lysates, polymerase chain reaction mixtures, and other reaction mixtures employed in recombinant DNA methodologies. Commercial products are available for the isolation of DNA. One example is the ILLUSTRA™ branded DNA isolation kits from GE Healthcare (Piscataway, N.J.). Many of these commercial products take advantage of the preferential and reversible binding of DNA to glass or other silicate, including glass fibre matrix, as well as silica membrane. Others employ ion-exchange resins that bind DNA.

Many analytical procedures and downstream applications in molecular biology require that the sample being cleaned of reaction components. For example, it is desirable to separate amplified PCR product from the reaction mixture (e.g. oligonucleotides, salts, nucleotides, polymerase enzymes, etc.) before subsequent applications. Dye-labeled nucleic acid probes also need to be separated from the un-incorporated dyes and enzymes before subsequent use in another procedure. Many separation means can be applied for this clean up process, such as silica membranes, glass fibre matrix columns, affinity chromatography and electrophoresis separation.

Few biologically active materials are sufficiently stable so that they can be isolated, purified, and then stored in solution at room temperature. Typically, biological reagents are stored at temperatures of 4° C., and especially enzymes are stored in glycerol at −20° C., or −70° C. They may be stored in bulk and then combined with other reagents before use. In preparing reagents for convenient and efficient testing of biological samples, it is frequently important to obtain dried reagents in uniform, discreet amounts which are stable at ambient temperature. It has been found that glass-forming filler materials effectively stabilize biological reagents in a dried, glassy state. For examples of glass-forming filler materials for stabilizing biological reagents see, for example, U.S. Pat. No. 5,098,893; U.S. Pat. No. 5,200,399 and U.S. Pat. No. 5,240,843.

Carbohydrates such as glucose, sucrose, maltose or maltotriose are an important group of glass-forming substances. Other polyhydroxy compounds can be used such as carbohydrate derivatives like sorbitol and chemically modified carbohydrates. Another important class of glass-forming substances is synthetic polymers such as polyvinyl pyrrolidone, polyacrylamide, or polyethyleneimine.

Further examples of glass-forming substances include sugar copolymers such as those sold by GE Healthcare under the registered trademark FICOLL™. FICOLL™ has molecular weights of 5,000 to 1,000,000 and as containing sucrose residues linked through ether bridges to bifunctional groups (U.S. Pat. No. 3,300,474). Such groups may be an alkylene of 2, 3 or more carbon atoms but not normally more than 10 carbon atoms. The bifunctional groups serve to connect sugar residues together. These polymers may, for example, be made by reaction of sugar with a halohydrin or bis-epoxy compound.

Stabilized biological materials in a glassy matrix of carbohydrate polymers, can be prepared, either by freeze-drying (Treml et al. U.S. Pat. No. 5,593,824; Franks and Hatley U.S. Pat. No. 5,098,893) or by vacuum drying (Walker et al. U.S. Pat. No. 5,565,318). Recently, improved manufacturing processes have been developed which allow effective freeze drying to produce stabilized biological materials in wells of a 96- or 384-well plate (WO2008/036544). These water-soluble reagents are convenient to use for complex molecular biology applications. This approach is particularly useful for reagent systems composed of enzymes, nucleotides and other components dispensed in single-use aliquots. Reconstitution of the glassy matrix delivers buffered enzymes for applications such as DNA amplifications and DNA sequencing.

There are currently a number of dried molecular biology products on the market. There are also a number of purification systems for macromolecules. However, there is a need to develop purification systems that reduces process steps and therefore minimizes human contact with the sample. There is also a need to develop systems that are ambient temperature stable for field use.

### SUMMARY OF THE INVENTION

According to the present invention there is provided a biological sample preparation system including ambient temperature stable reagent mixture and a separation filter or membrane. In particular, it includes a dried reagent in a glassy, porous state, on top of a separation column, whereby sample preparation is streamlined and simplified. Also provided are methods of making and using the system. A kit for preparing a biological sample is also provided.

In a first embodiment, the invention provides a sample preparation system for a biological sample, comprising: a dried reagent mixture for processing the biological sample; and means for separating components of the biological sample. The dried reagent mixture, when rehydrated, is used to process the biological sample, and the separation means is capable of separating components of interest from the biological sample. Usually, the dried reagent mixture includes at least one reagent that is temperature sensitive in an aqueous solution and is ambient temperature stable in the dried mixture.

It is preferable to have the dried reagents prepared by lyophilization. A wide variety of reagents are shown to retain activity, once reconstituted from a lyophilized state, and works well in the sample preparation system according to embodiments of the current invention. One example includes reagents for the lysis of cells, such as proteinase K. Another example includes reagents for labeling a biological substrate such as dyes suitable for labeling a macromolecule. Other examples include reagents for both nucleic acid labeling and amplification.

There are a number of different separation devices that are suitable for the sample preparation systems of the invention. In one instance, the separation device is a glass fibre matrix column. In another instance, the separation device is any dry column, including a column with a rehydrable matrix. Alternatively, the separation device is a silica membrane column. Preferably the dried reagent mixture is on top of, and in contact with the separation device.

In a second embodiment, the invention provides a sample preparation system for parallel processing of multiple biological samples, comprising: multiple individual sample preparation system as described in the first embodiment, arranged in a predetermined format. Preferably, the parallel sample preparation system is in a 96-well plate format.

In another embodiment, the invention provides a method for making the biological sample preparation system, comprising the steps of: (a) providing an aqueous solution of at least one buffered reagent; (b) mixing a glass forming filler material with the buffered reagent solution to form a mixture wherein the concentration of the filler material is sufficient to facilitate formation of a glassy, porous composition; (c) providing a dry depth column for separating components of the biological sample; (d) dispensing a predetermined amount of the mixture from step (b) into the column; and (e) drying the mixture in the column to form a dried reagent preparation; wherein the reagent preparation is water soluble and has a Tg sufficient for room temperature stability.

In yet another embodiment, the invention provides a method for making the biological sample preparation system, comprising the steps of: (a) providing an aqueous solution of at least one buffered reagent; (b) mixing a glass forming filler material with said buffered reagent solution to form a mixture wherein the concentration of the filler material is sufficient to facilitate formation of a glassy, porous composition; (c) dispensing a predetermined amount of the mixture from step (b) into a container; (d) drying the mixture in the container to form a dried reagent preparation, wherein the reagent preparation is water soluble and has a Tg sufficient for room temperature stability; and (e) transferring the dried reagent preparation to a dry depth column to complete the biological sample preparation system.

In still another embodiment, the invention provides a method for preparing a biological sample, comprising the steps of: (a) providing a sample preparation system according to the first embodiment of the invention; (b) reconstituting the dried reagent mixture with an aqueous solution; (c) combining the biological sample with reconstituted reagent mixture; (d) incubating combination to process the biological sample; and (e) separating components of the biological sample. In certain applications, the dried reagent mixture includes reagents for the lysis of cells. For other applications, the dried reagent mixture includes reagents for labeling a biological substrate, such as a nucleic acid sample. The dried reagent mixture could also contain reagents for nucleic acid amplification.

In another embodiment, the invention provides a kit for processing a biological sample, comprising a sample preparation system according to the previous embodiments and a user manual.

### DESCRIPTION OF THE DRAWINGS

FIG. 1 is a work flow comparison for genomic DNA purification between the prior art spin column format from GE Healthcare with that of the current invention.

FIG. 2 shows, on the left, the stabilization on top of a column, cell lysis reagents with different amounts of TWEEN® 20 (0.1%, 1%, 2% and 5%); and on the right, DNA isolated using a column prepared with lyophilized reagents with 5% TWEEN® 20, as compared to the ‘wet’ form of lysis reagents.

FIG. 3 shows lyophilized DNA labeling reagents in glass fibre matrix columns. The columns are capped so that the reagents are kept from exposure to moisture.

FIG. 4 shows that similar to the conventional DNA labeling reagents, lyophilized DNA labeling reagents label DNA in a similar fashion.

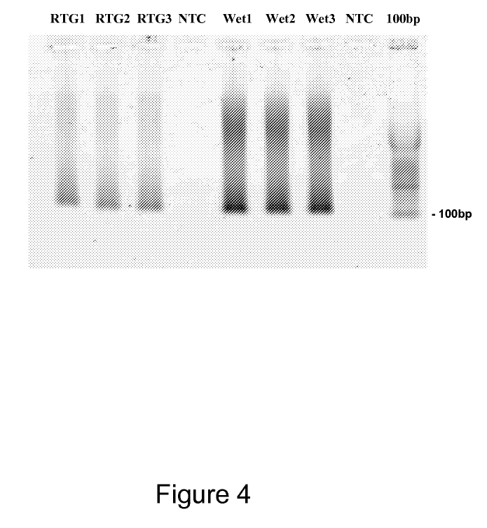

### DETAILED DESCRIPTION OF THE INVENTION

We provide a novel sample preparation system for biological samples. Pre-formulated temperature sensitive reagents for sample processing are lyophilized and combined with a separation device such as a membrane filter. The lyophilized reagents are stable at ambient temperature, thus this combined system is ideal for field applications. The system also reduces sample processing steps and simplifies the work flow for many molecular biology processes, and it is especially advantageous for applications where minimum human contact is desirable, i.e. the handling of human body fluids or pathogen.

### The Reagents

Many biological reagents are ambient temperature stable when lyophilized by the method of the present invention. The biological reagent compositions of the present invention are particularly suitable for performing a wide variety of analytical procedures which are beneficially or necessarily performed on a variety of biological samples, whether purified or not. The analytical procedures will generally require that the sample be combined with one or more reagents.

One category of biological reagents to which the present invention is applicable is protein and peptides, including derivatives thereof such as glycoproteins. Such proteins and peptides may be any of: enzymes, transport proteins (for example hemoglobin, immunoglobulins, hormones, blood clotting factors and pharmacologically active proteins or peptides).

Another category of biological reagents to which the invention is applicable comprises nucleosides, nucleotides (such as deoxynucleotides, ribonucleotides and dideoxynucleotides), dinucleotides, oligonucleotides and also enzyme cofactors, whether or not these are nucleotides. Enzyme substrates in general are also biological reagents to which the invention may be applied.

Another development of this invention is to store more than one reagent of a reacting system in a lyophilized state. In fact, it is envisioned that the majority of the applications would benefit from a lyophilized reaction system, rather than individual components. This system normally comprised biological reagents, chemicals and buffer components which will be required to be used together in, for example, an assay or a diagnostic kit. Ideally, all the components necessary for a certain assay are included at the appropriate proportion such that only water is needed to reconstitute the reaction system prior to an assay.

Storing the reagents in a single glassy preparation provides them in a convenient form for eventual use. For instance, if an assay requires a combination of a substrate or cofactor and an enzyme, two or all three could be stored in a dried lyophilized state, in the required concentration ratio and be ready for use in the assay.

If multiple reagents are stored, they may be mixed together in an aqueous emulsion and then incorporated together into a glass. Alternatively, they may be incorporated individually into separate glasses which are then mixed together.

When multiple reagents are stored as a single composition (which may be two glasses mixed together) one or more of the reagents may be a protein, peptide, nucleoside, nucleotide, or enzyme cofactor. It is also possible that the reagents may be simpler species. For instance, a standard assay procedure may require pyruvate and NADH to be present together. Both can be stored alone with acceptable stability. However, when brought together in an aqueous solution they begin to react. If put together in required proportions in the glassy lyophilized state, they do not react and the glass can be stored. By react we mean any biochemical reaction.

The preferred biological reagents of the present invention are enzymes and cofactors that provide a reagent system to isolate, label, detect, amplify, modify or sequence nucleic acids. Such enzymes include but are not limited to proteinases, DNA polymerases (e.g., Klenow), T7 DNA polymerase or various thermostable DNA polymerases such as Taq DNA polymerase; AMV or murine reverse transcriptase, Phage Phi29 DNA polymerase, and restriction enzymes. Cofactors include nucleotides, oligonucleotides, DNA, RNA, required salts for enzyme activity (e.g., magnesium, potassium and sodium), and salts required for buffer capacity. Buffer salts provide a proper pH range and aid stability. Some buffers which may be used include Tris pH 7.6-8.3.

Any potential biological reagents may be evaluated using a protocol similar to Example 1, infra. Thus, suitable biological reagents are rendered stable in the lyophilized state as determined by a functionality test like that in Example 1.

### The Separation Device

A suitable separation device for the current sample preparation system needs to contain a compartment that is moisture free. The biological reagents lyophilized are stored in this compartment prior to reconstitution and reaction with the sample of interest.

There are many separation devices to choose from. It is desirable to choose a device that is effective in separating the macromolecules of interest which could also withstand the lyophilization process. Such a device enables lyophilization of a reagent mixture within the device. Alternatively, one could lyophilize the reagents separate from the separation device and then introduce the lyophilized reagents into the separation device. This is less desirable as it is cumbersome. However, it is suitable for devices that could not withstand the lyophilization condition.

A glass fibre matrix column such as one in the ILLUSTRA™ blood genomicPrep Mini Spin Kit (GE Healthcare) is a suitable device for nucleic acid purification. Our tests show that it is able to withstand the lyophilization process. Another suitable example is a silica membrane based column. For other separation devices, such as chromatography or gel filtration columns, a successful integration with lyophilized reagents (to form a sample preparation system) requires the presence of a moisture barrier in the column that prevents the moisture from “wetting” the dried reagent, which rendering the reagents unstable. In such cases, the reagents are lyophilized first independent of the separation device, and then combined to form the system.

In certain embodiments, the columns are provided individually. In other embodiments, the columns are molded into strips of 8 to 12 columns, preferably sized to accommodate a standard 96-well sample dish. Individual columns can be separated from such strips for single-sample applications. In such embodiments, the columns are preferably molded from a material that can be easily broken, including but not limited to plastics such as styrene, acrylic, polypropylene, polycarbonate, polysulfone, and the like.

### The Lyophilization Process

The lyophilization process is similar to that described in WO2008/036544, the disclosure of which is hereby incorporated by reference in its entirety. A typical process is described in detail in Example 1, infra. The following provides a brief summary of the process.

Glass-Forming Filler Material: Examples of glass forming filler materials which may be used in the present invention include carbohydrates such as FICOLL™, sucrose, glucose, trehalose, melezitose, DEXTRAN™, and mannitol; proteins such BSA, gelatin, and collagen; and polymers such as PEG and polyvinyl pyrrolidone (PVP). The glass forming filler materials are preferably FICOLL™ polymer, BSA, sucrose, DEXTRAN™, or combinations thereof. A most preferred glass forming filler material for use in the present invention is FICOLL™ polymer.

Formulation: The formulation of a high viscosity mixture of biological reagent, glass forming filler material, and water is determined by an iterative process. First, one determines final as used concentrations desired of the system. Each biological reagent may have different formulations. Secondly, these concentrations are converted to a weight/dose basis for solids and a volume/dose basis for liquids. Third, an initial value is chosen for the percent solids concentration of the high viscosity mixture and the desired mixture volume. A 22.5-25% solids concentration has been shown to work well. Fourth, one calculates the number of doses that can be made using the grams of glass forming material per dose from the second step. Fifth, using the number of doses and the weight per dose ratios from the second step, one determines the weights in volumes of the other components. Finally, using the weights and volumes determined in the fifth step, one calculates the percent solids of the final mixture. If the final percent solids of the mixture are out of the desired range, one repeats the third through sixth steps with another initial value until the final value is in the correct range. Any potential glass forming material may be evaluated using a protocol according to the iterative process described above. Thus, a suitable glass forming material produces a reagent preparation having an acceptable hardness, size, shape, T_{g}, porosity, solubility, and stability.

Mixing and Dispensing: A typical formulation (using DNA labeling formulation as the example) is made as shown in Example 1. Note that all reagents used are typically autoclaved or filter sterilized (preferably a 0.25 μm filter) before use. Formulations are made and stored on ice until dispensed. Just before use, d(N)₉ primer are added. Before adding to the formulation, the primer should be heated at 65° C. for 7 minutes and quickly cooled on ice. The Klenow DNA polymerase could be added to the bulk formulation or individually after the formulation is dispersed to the column or container. Prior to dispersion, the final volume should be brought to pre-calculated amount with sterile water.

For separation devices such as the glass fibre matrix column, the formulation is added to the top of the column, followed by the addition of Klenow enzyme. If the formulation is to be lyophilized separate from the device, then the enzyme is added just prior to dispersion of the formulation. The formulation can be dispersed into liquid nitrogen (U.S. Pat. No. 5,593,824) or collection tubes such as individual wells of a 96 well plate. The dispensed solution is dried by the protocols described later.

Drying Process: The mixture dispensed can be dried by vacuum drying, freeze-drying or lyophilization. A suitable drying program produces a reagent preparation having an acceptable hardness, size, shape, T_{g}, porosity, solubility, and stability. A preferred method of drying is by way of lyophilization. The dispensed reagents are successfully dried on top of a glass fibre matrix column or in a 96-well polystyrene plate. When the glass fibre matrix column or the 96-well polystyrene plate was placed in direct contact with a metal plate holder, the drying process works better. Direct contact of the outside wall of a polystyrene well (tube) with the metal plate holder enhances the metal shelf contact area, which in turn achieves a better heat transfer to the samples. A preferred lyophilization profile is shown below in Example 1.

Storage: The dried reagent preparations can be stored in the column when properly sealed. Sealing of the plate or mould can be achieved by: lid, tape, heat activated tape etc. In one embodiment of the invention, sealing of the plates is achieved by heat activation sealing using ABgene®'s Thermo-Seal Heat Seals and Easy Peel Heat Seals.

We successfully prepared stable biological reagents on top of a sample separation column. Our technology allows the combination of a macromolecular separation device with assay reagents at ambient temperature, thus provides much simplified sample processing protocol, and eliminates the need of low temperature storage of many temperature sensitive reagents. These systems can be used for a variety of molecular biology applications, including but not limited to sample purification, labeling, detection, nucleic acid amplification and cDNA synthesis applications.

A reagent preparation of the present invention has a glass transition temperature (T_{g}) of at least 10° C. A typical T_{g} of the reagent preparation is 40° C. A T_{g} of at least 40° C. will guarantee stability at room temperature (22° C.). A preferred T_{g} is 45° C. or higher. The glass transition temperature is the temperature above which the viscosity of a glassy material drops rapidly and the glassy material turns into a rubber, then into a deformable plastic which at even higher temperatures turns into a fluid.

Our novel sample preparation system offers several advantages. The lyophilized reagent mixture usually contains all the necessary components for a certain assay application. This eliminates the need of making and mixing the reagent components before starting each assay. The assay workflow is simplified, thus less process related error is likely to happen. It also offers increased reproducibility and reliability, as it reduced risk of contamination and errors. In addition, the compositions made are stable at ambient temperature. This saves cost on shipping (no dry-ice shipping), eliminates the need for freezer storage and shortens the reagent preparation time (no thawing).

We show below exemplary sample preparation systems made according to embodiments of the invention. One system includes stabilized reagents for DNA labeling and a purification column for separating the unlabelled components from the labeled probes. Another system includes lyophilized lysis buffer for lysing cells and a column for the purification of nucleic acids from the lysed cells. Yet another example shows one can combine isothermal nucleic acid application and product purification in one device. However, there are many other possibilities to combine a biological assay system with a assay purification system according to the teachings of the invention. Alternatively, it is envisioned that the various systems could be used consecutively. For example, DNA could be purified from cells using one such system that combines cell lysis and DNA purification, then the resultant isolated DNA could be amplified and purified using a different system that combines isothermal amplification with DNA purification. With combinations of such systems, researcher's contact with the input material and intermediates can be greatly reduced.

### EXAMPLES

The present examples are provided for illustrative purposes only, and should not be construed as limiting the scope of the present invention as defined by the appended claims. All references given below and elsewhere in the present specification are hereby included herein by reference.

### Example 1

### Preparation and Use of Dried Reagent Mixture on Top of Glass Fibre Matrix Columns for DNA Purification

In order to purify DNA from cells, one has to first break apart the cells. Then DNA is selectively separated from other macromolecules. We developed a protocol for generating lyophilized lysis buffer on top of a DNA separation column. We show that the dried buffer, when reconstituted in water, is effective in lysing the cells. This lyophilized reagent/separation column combination is successful in purifying genomic DNA from human blood.

Nucleic acid purification columns like glass fibre matrix columns from the ILLUSTRA™ blood genomicPrep Mini Spin Kit (GE Healthcare) are designed to isolate genomic DNA efficiently in a short-period of time. We successfully lyophilized the lysis buffer containing Tris, EDTA, Guanidine-HCl, TWEEN® 20 and Proteinase K on the top of the spin columns. The buffer can be reconstituted immediately prior to DNA isolation. Sample blood can be added directly to the column for processing which reduces processing time and eliminates the necessity to store the reagents at colder temperatures.

Lyophilized reagent mixture was made according to the following protocol. Lysis buffer was prepared as 50 mM Tris-HCl (pH 7.0), 10 mM EDTA, 7 M Guanidine-HCl and varying amount of TWEEN® 20 (0.1%, 1%, 2% and 5% respectively). The above buffer was mixed with stabilizers (10% Melezitose, 6.25% FICOLL™ 70 and 6.25% FICOLL™ 400). Proteinase K was prepared as 20 mg/ml solution. First, 200 μl of each lysis buffer with the stabilizer, respectfully, was added to the top of a spin column from the ILLUSTRA™ blood genomicPrep Mini Spin Kit. Then 20 μl of 20 mg/ml Proteinase K was added to top of each spin column. The columns were kept in a metal holder and the reagents were lyophilized using a Vertis Freeze-dryer, according to the drying conditions shown in Table 1. The addition of stabilizers caused some “foaming” during the drying process, however the performance of the dried reagents were not affected. The “foaming” should be corrected by optimizing the initial freezing and primary drying conditions.

TABLE 1
Drying conditions

| Temperature (° C.) | Vacuum (mTorr) | Time (min) | Comment |
|---|---|---|---|
| −45 | atm | 120 | Hold |
| −45 | 100 | 600 | Hold |
| −36 | 100 | 250 | Ramp |
| −36 | 100 | 300 | Hold |
| 0 | 100 | 400 | Ramp |
| 0 | 100 | 300 | Hold |
| 28 | 100 | 200 | Ramp |
| 28 | 100 | 240 | Hold |

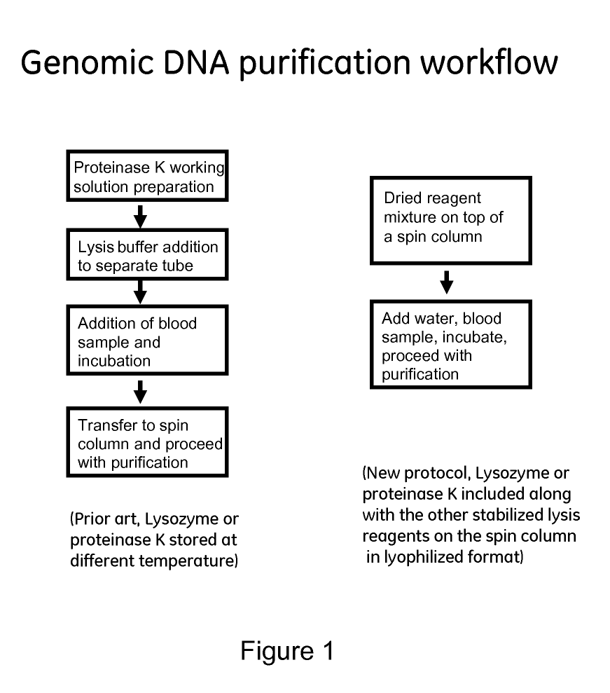

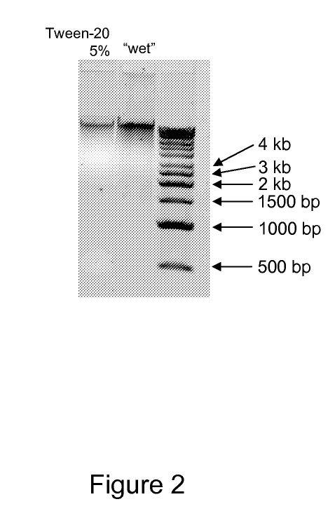

We tested the stability of the lyophilized reagents by their ability to purify genomic DNA from human blood. To use the lyophilized reagents, they were first reconstituted with 200 μl of water. This was followed by the addition of 100 μl of blood sample. The content was mixed with pipette tip and incubated at room temperature for 10 minutes. Genomic DNA was isolated from the lysed sample following further wash steps and elution according to protocol of the ILLUSTRA™ blood genomicPrep Mini Spin Kit. As a control, we also isolated genomic DNA from the same blood source in parallel, following the regular protocol presented in the Kit. FIG. 1 presents a workflow comparison between the prior art protocol and the simplified protocol according to the present invention. Purified genomic DNA was analyzed on a 0.8% agarose gel. Human Genomic DNA was successfully purified from lyophilized reagents with the stabilizer (FIG. 2).

### Example 2

### Preparation and Use of Dried Reagent Mixture on Top of a Glass Fibre Matrix Column for DNA Labeling

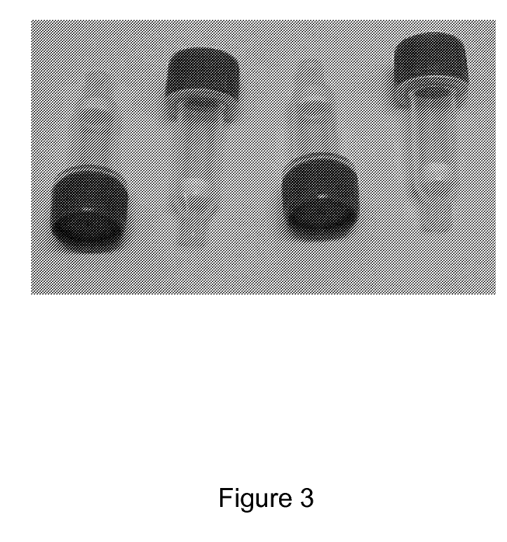

Mixtures containing enzymes and other reactants necessary for labeling DNA molecules were stabilized on top of the glass fibre matrix in a spin column (GFX™ PCR gel band purification kit, GE Healthcare, Piscataway, N.J.). Ten μl volume of the DNA labeling reagent containing the stabilizers (Table 2) was dispensed on top of the glass fibre matrix, and lyophilized in VirTis freeze dryer according to the protocol in Table 1. The columns containing lyophilized reagents were capped to prevent the reagent from exposure to moisture (FIG. 3).

TABLE 2
Labeling reagent

| Tris pH 7.5 | 50 mM |
|---|---|
| MgCl2 | 50 mM |
| DTT | 50 mM |
| NaCl | 250 mM |
| d(N)9 | 4 u/ml |
| BSA | 2.5 mg/ml |
| Exo-Klenow | 20 U |
| dNTPs | 400 uM |
| CY ™ 3 dCTP | 100 uM |
| FICOLL ™ 400 | 7.5% |
| FICOLL ™ 70 | 7.5% |
| Melezitose | 10% |

The functionality of the dried enzymes and reagents was tested by adding 50 μl of (1 μg) heat denatured lambda DNA to the spin column containing the dried reagents, pipetting up and down for a few times, and incubating at 37° C. in an incubator for 60 minutes. For the purification of the labeled probe from the unincorporated CY™3 dCTP, 500 μl of capture buffer (GFX™ PCR gel band purification kit) was added to the reaction mixture and mixed thoroughly by pipetting up and down a few times. The samples were filtered by centrifugation at 13,000 rpm for 30 seconds and discarded the filtrate. The samples were washed with 500 μl of wash buffer and the column was transferred to a fresh collection tube. Fifty micro liters of TE buffer was added to the centre of the column. The column was allowed to stand in dark at room temperature for 1-2 minutes and the samples were eluted by centrifuging at 13000 rpm for 1 minute. The samples were quantified by measuring the absorbance at different wavelengths (260, 320, 550 and 650 nM, Table 3). The labeled products were run on 0.8% agarose gel along with labeled products from “wet” control experiments in triplicates. Labeled products were produced by the lyophilized reagents compared to the non-stabilized “wet” control experiments. It is expected that further optimization of the lyophilization process will improve the labeling yields.

TABLE 3
DNA yield, amount of fluorescent labeled dye and nucleotide/dye ratio

| | | | | | Total | |
|---|---|---|---|---|---|---|
| | | | | Total | Dye | |
| | A260 | Probe | DNA | Nuc's | Inc'd | Nucleotides/ |
| Name | (Net) | Vol (μl) | (ng/μl) | (pmol) | (pmol) | Dye |
| RTG-1 | 0.281 | 60 | 14.05 | 2594 | 30 | 88 |
| RTG-2 | 0.188 | 60 | 9.39 | 1733 | 18 | 96 |
| RTG-3 | 0.186 | 60 | 9.31 | 1718 | 7 | 262 |
| RTG-NTC | 0.009 | 60 | 0.47 | 87 | 0.20 | 434 |
| Wet-1 | 0.766 | 60 | 38.32 | 7074 | 59 | 120 |
| Wet-2 | 0.670 | 60 | 33.5 | 6184 | 41 | 150 |
| Wet-3 | 0.721 | 60 | 36.06 | 6657 | 39 | 170 |
| Wet-NTC | 0.006 | 60 | 0.28 | 52 | 3 | 16 |

### Example 3

### Preparation of Dried DNA Amplification Reagent Mixture on Top of a Glass Fibre Matrix Column

Phi29 DNA polymerase is widely used for whole genome amplification as well as rolling circle amplification. To provide a sample preparation system that combines DNA amplification and purification, this enzyme is lyophilized on top of a glass fibre matrix column, in a formulation that enables whole genome amplification.

GENOMIPHI™ HY DNA Amplification Kit (GE Healthcare) contains all the components necessary for whole genome amplification by isothermal strand displacement amplification. The starting material for GENOMIPHI™ reactions can be purified DNA or non-purified cell lysates. Microgram quantities of DNA can be generated from nanogram amounts of starting material in only a few hours. Typical DNA yields from a GENOMIPHI™ HY reaction are 40-50 μg per 50 μl reaction, with an average product length of greater than 10 kb. DNA replication is extremely accurate due to the proofreading 3′-5′ exonuclease activity of the enzyme.

GENOMIPHI™ reaction mixture is prepared including Phi29 DNA polymerase, random hexamers, dNTPs and the GENOMIPHI™ HY reaction buffer along with the stabilizers FICOLL™ 70, FICOLL™ 400, Melezitose and BSA, as a 2× mix. Ten μl volume aliquots of the mixture are dispensed into a glass fibre matrix column. The dispensed products are lyophilized using VirTis freeze-drier. The dried products are stored at room temperature or at 40° C. for 35 days. Whole genome amplification is performed with these products using human genomic DNA as template material, with a 90 minutes amplification reaction at 30° C. Amplified DNA is purified following the protocol of GFX™ PCR gel band purification kit (GE Healthcare). It is expected that greater than 4 μg of DNA should be produced in 90 minutes from 10 ng template. Using PICOGREEN® assay, amplification is detected with lyophilized reagent. Phi29 DNA polymerase was successfully stabilized in lyophilized format.

While the preferred embodiment of the present invention has been shown and described, it will be obvious in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A sample preparation system for a biological sample, comprising:
(a) a dried reagent mixture for processing said biological sample; and
(b) means for separating components of said biological sample;
wherein said dried reagent mixture, when rehydrated, is used to process said biological sample, and wherein said separation means is capable of separating components of interest from said biological sample.

2. The sample preparation system of claim 1, wherein said dried reagent mixture includes at least one reagent which is temperature sensitive in an aqueous solution and is ambient temperature stable in said dried mixture.

3. The sample preparation system of claim 1, wherein said dried reagent mixture includes reagents for lysis of cells.

4. The sample preparation system of claim 1, wherein said dried reagent mixture includes reagents for labeling a biological substrate.

5. The sample preparation system of claim 1, wherein said biological sample includes nucleic acid substrate and said dried reagent mixture includes reagents for labeling said nucleic acid substrate.

6. The sample preparation system of claim 1, wherein said biological sample includes nucleic acid substrate and said dried reagent mixture includes reagents for nucleic acid amplification.

7. The sample preparation system of claim 1, wherein said separation means includes a silica membrane.

8. The sample preparation system of claim 1, wherein said separation means includes a dry depth column.

9. The sample preparation system of claim 8, wherein said dry depth column is a glass fibre matrix column.

10. The sample preparation system of claim 1, wherein said dried reagent mixture is on top of, and in contact with said separation means.

11. A sample preparation system for parallel processing of multiple biological samples, comprising: multiple individual systems of claim 1, arranged in a predetermined format.

12. The sample preparation system for parallel processing of multiple biological samples of claim 11 in a 96-well plate format.

13. A kit for processing a biological sample, comprising a sample preparation system of claim 1 and a user manual.

14. A kit for parallel processing of multiple biological samples, comprising a sample preparation system of claim 11 and a user manual.

15. A method for making a biological sample preparation system, comprising the steps of:
(a) providing an aqueous solution of at least one buffered reagent;
(b) mixing a glass forming filler material with said buffered reagent solution to form a mixture wherein the concentration of the filler material is sufficient to facilitate formation of a glassy, porous composition;
(c) providing a dry depth column for separating components of said biological sample;
(d) dispensing a predetermined amount of the mixture from step (b) into the column; and
(e) drying the mixture in said column to form a dried reagent preparation;
wherein the reagent preparation is water soluble and is room temperature stable.

16. A method for making a biological sample preparation system, comprising the steps of:
(a) providing an aqueous solution of at least one buffered reagent;
(b) mixing a glass forming filler material with said buffered reagent solution to form a mixture wherein the concentration of the filler material is sufficient to facilitate formation of a glassy, porous composition;
(c) dispensing a predetermined amount of the mixture from step (b) into a container;
(d) drying the mixture in said container to form a dried reagent preparation, wherein the reagent preparation is water soluble and is room temperature stable; and
(e) transferring the dried reagent preparation to a dry depth column to complete the biological sample preparation system.

17. A method for preparing a biological sample, comprising:
(a) providing a sample preparation system of claim 1;
(b) reconstituting the dried reagent mixture with an aqueous solution;
(c) combining said biological sample with said reconstituted reagent mixture;
(d) incubating said combination to process said biological sample; and
(e) separating components of said biological sample.

18. The method for preparing a biological sample of claim 17, wherein said dried reagent mixture includes reagents for lysis of cells.

19. The method for preparing a biological sample of claim 17, wherein said dried reagent mixture includes reagents for labeling a biological substrate.

20. The method for preparing a biological sample of claim 17, wherein said biological sample includes nucleic acid substrate and said dried reagent mixture includes reagents for labeling said nucleic acid substrate.

21. The method for preparing a biological sample of claim 17, wherein said biological sample includes nucleic acid substrate and said dried reagent mixture includes reagents for nucleic acid amplification.

22. The method for making a biological sample preparation system of claim 15, wherein said drying step is performed while the column remains in contact with a metal column holder.

23. The method for making a biological sample preparation system of claim 16, wherein said drying step is performed while the container remains in contact with a metal holder.
